(19) United States
(12) Patent Application Publication
(10) Pub. No.: US 2026/0108164 A1
(43) Pub. Date: Apr. 23, 2026

(54) METHOD FOR CONTROLLING HEART SOUND DATA DETECTING DEVICE, DEVICE, EARPLUG, AND STORAGE MEDIUM

(71) Applicant: GOERTEK INC., Weifang (CN)

(72) Inventors: Chenghao SUI, Weifang (CN); Junjie PAN, Weifang (CN); Yaocheng LIU, Weifang (CN); Yanan ZHOU, Weifang (CN)

(21) Appl. No.: 19/422,631

(22) Filed: Dec. 17, 2025

Related U.S. Application Data
(63) Continuation of application No. PCT/CN2025/093118, filed on May 7, 2025.

(30) Foreign Application Priority Data
Mar. 27, 2024 (CN) 202410850615.7

(51) Int. Cl. A61B 5/024; G16H 40/67; G16H 50/30

(52) U.S. Cl. CPC A61B 5/02438; G16H 40/67; G16H 50/30

(57) ABSTRACT

A method for controlling heart sound data detecting device includes: obtaining a first heart sound data acquired by a first earbud and a second heart sound data synchronously acquired by a second earbud; analyzing the first heart sound data to obtain a first user state analysis result and analyzing the second heart sound data to obtain a second user state analysis result; and in response to that the first user state analysis result and the second user state analysis result meet a consistency requirement, and outputting the first user state analysis result and/or the second user state analysis result.

### CROSS-REFERENCE TO RELATED APPLICATIONS

This application is a continuation application of International Application No. PCT/CN2025/093118, filed on May 7, 2025, which claims priority to Chinese Patent Application No. 202410850615.7, filed on Jun. 27, 2024. The disclosures of the above applications are incorporated herein by reference in their entireties.

### TECHNICAL FIELD

The present application relates to the technical field of data detecting, and in particular to a method for controlling a heart sound data detecting device, a device, an earplug, and a storage medium.

### BACKGROUND

Heart sound data is the sound produced by the vibrations of the heart during contraction and relaxation, reflecting the condition of the heart and cardiovascular system during contraction and relaxation, including various physiological and physical information generated by the heart itself and its interaction with various surrounding tissues. The acquiring heart sound data typically requires specialized medical device or heart monitoring devices, such as a stethoscope or electronic heart stethoscope.

In order to improve the convenience of acquiring heart sound data, smartwatches can be used to acquire heart sound data. The principle is that that a sensor on a smartwatch that comes into contact with the skin emits a beam of light that hits the skin. Because blood absorbs light of a specific wavelength, this wavelength is absorbed in large quantities each time the heart pumps blood, thus allowing the heartbeat to be determined. Therefore, the measurement results will vary if the watch is worn in different positions or tightness, making it difficult for users to determine the reliability of the measurement results output by the smartwatch.

The above content is only used to assist in understanding the technical solution of the present application and does not constitute an admission that the above content is related art.

### SUMMARY

The main purpose of the present application is to provide a method for controlling a heart sound data detecting device, a device, an earbud, and a storage medium, aiming to solve the technical problem of insufficient reliability in heart sound data detecting.

In order to achieve the above purpose, the present application provides a method for controlling a heart sound data detecting device, including:
obtaining a first heart sound data acquired by a first earbud and a second heart sound data synchronously acquired by a second earbud;
analyzing the first heart sound data to obtain a first user state analysis result and analyzing the second heart sound data to obtain a second user state analysis result; and
in response to that the first user state analysis result and the second user state analysis result meet a consistency requirement, outputting the first user state analysis result and/or the second user state analysis result

In an embodiment, the heart sound data detecting device is a master control device, and the obtaining the first heart sound data acquired by the first earbud and the second heart sound data acquired synchronously by the second earbud includes:
sending an instruction for starting acquisition to the first earbud and receiving the first heart sound data sent by the first earbud based on the instruction for starting acquisition;
sending an instruction for ending acquisition to the first earbud, disconnecting from the first earbud and establishing a connection with the second earbud; and
receiving the second heart sound data sent by the second earbud based on the instruction for ending acquisition.

In an embodiment, the heart sound data detecting device is a cloud device, and the obtaining the first heart sound data acquired by the first earbud and the second heart sound data acquired synchronously by the second earbud includes:
receiving the first heart sound data acquired by the first earbud and the second heart sound data acquired synchronously by the second earbud from a master control device; and
the in response to that the first user state analysis result and the second user state analysis result meet the consistency requirement, outputting the first user state analysis result and/or the second user state analysis result includes:
in response to that the first user state analysis result and the second user state analysis result meet the consistency requirement, sending the first user state analysis result and/or the second user state analysis result to the master control device.

In an embodiment, after the analyzing the first heart sound data to obtain the first user state analysis result and analyzing the second heart sound data to obtain the second user state analysis result, the method further includes at least one of the following steps:
in response to that the first user status analysis result and the second user status analysis result do not meet the consistency requirement, outputting an abnormality alert; and
in response to detecting that a user triggers a re-acquisition instruction and/or a re-inspection process based on the abnormality alert, jumping to execute the obtaining the first heart sound data acquired by the first earbud and the second heart sound data synchronously acquired by the second earbud.

In an embodiment, before the analyzing the first heart sound data to obtain the first user state analysis result and analyzing the second heart sound data to obtain the second user state analysis result, the method further includes:
extracting a first key feature of the first heart sound data and a second key feature of the second heart sound data;
calculating a similarity between the first key feature and the second key feature according to a similarity algorithm; and
filtering noise from the first heart sound data and the second heart sound data according to the similarity and a preset similarity threshold.

In addition, in order to achieve the above purpose, the present application also provides a method for controlling a heart sound data detecting device, including:
in response to that a first earbud detects that an acquisition process is triggered, obtaining a delayed acquisition time;
sending the delayed acquisition time to a second earbud; and
in response to reaching the delayed acquisition time, performing an acquisition operation to obtain a first heart sound data.

In an embodiment, the method further includes:
in response to that the second earbud receives the delayed acquisition time, determining a transmission delay;
determining a target delay based on the delayed acquisition time and the transmission delay; and
in response to reaching the target delay, performing the acquisition operation to obtain second heart sound data.

In addition, in order to achieve the above purpose, the present application also provides a heart sound data detecting device, the heart sound data detecting device includes: a memory, a processor and a computer program stored in the memory and executable on the processor, the computer program is configured to implement the method for controlling the heart sound data detecting device as described above.

In addition, in order to achieve the above purpose, the present application also provides an earplug, the earplug includes: a memory, a processor and a computer program stored in the memory and executable on the processor, the computer program is configured to implement the method for controlling the heart sound data detecting device as described above.

In addition, in order to achieve the above purpose, the present application also provides a non-transitory computer-readable storage medium, a computer program is stored on the non-transitory computer-readable storage medium, and when the computer program is executed by a processor, the method for controlling the heart sound data detecting device as described above is implemented.

One or more technical solutions proposed in the present application have at least the following technical effects.

By simultaneously acquiring a heart sound data with a first earbud and a second earbud, dual verification of the same physiological signal is achieved. Only when the analysis results of the two are consistent will the data be considered valid, this mechanism ensures data reliability. Furthermore, the user only needs to wear the earbuds throughout the entire detecting process, eliminating the need to visit a medical facility. This ease of wear and operation greatly enhances the convenience of heart sound data detecting.

### BRIEF DESCRIPTION OF THE DRAWINGS

The drawings incorporated in the specification form a part of the specification and shows embodiments corresponding to the present application, and are used to explain the principle of the present application together with the specification.

In order to illustrate the technical solutions in the embodiments of the present application or in the related art more clearly, the following briefly introduces the accompanying drawings required for the description of the embodiments or the related art. Obviously, the drawings in the following description are only part of embodiments of the present application. For those skilled in the art, other drawings can also be obtained according to the structures shown in these drawings without any creative effort.

FIG. 1 is a flow chart a method for controlling a heart sound data detecting device according to an embodiment of the present application.

FIG. 2 is a schematic diagram a heart sound data of the method for controlling the heart sound data detecting device according to an embodiment of the present application.

FIG. 3 is an interaction diagram of the method for controlling the heart sound data detecting device according to an embodiment of the present application.

FIG. 4 is a flow chart of the method for controlling the heart sound data detecting device according to an embodiment of the present application.

FIG. 5 is a flow chart of the method for controlling the heart sound data detecting device according to an embodiment of the present application.

FIG. 6 is a flow chart of the method for controlling the heart sound data detecting device according to another embodiment of the present application.

FIG. 7 is a signaling flow chart of the method for controlling the heart sound data detecting device according to another embodiment of the present application.

FIG. 8 is a flow chart of the method for controlling the heart sound data detecting device according to another embodiment of the present application.

FIG. 9 is a signaling flow of the method for controlling the heart sound data detecting device according to another embodiment of the present application.

FIG. 10 is a flow chart of the method for controlling the heart sound data detecting device according to another embodiment of the present application.

FIG. 11 is a flow chart of the method for controlling the heart sound data detecting device according to another embodiment of the present application.

FIG. 12 is a simplified flow chart of the method for controlling the heart sound data detecting device according to another embodiment of the present application.

FIG. 13 is a schematic diagram of a device structure of a hardware operating environment involved in the method for controlling the heart sound data detecting device according to an embodiment of the present application.

The objectives, features, and advantages of the present application will be further explained in conjunction with the embodiments and with reference to the accompanying drawings.

### DETAILED DESCRIPTION OF THE EMBODIMENTS

It should be understood that the specific embodiments described herein are merely used to explain the technical solutions of the present application and are not intended to limit the present application.

In order to better understand the technical solution of the present application, a detailed description will be given below in conjunction with the accompanying drawings and specific implementation methods.

The main solution of the embodiment of the present application is: obtaining the first heart sound data and the second heart sound data acquired synchronously by the first earplug and the second earplug, and obtaining the corresponding first user status analysis result and second user status analysis result through analysis. If the two are consistent, they are directly output.

In related art, heart sound data can be acquired using a variety of methods.

Firstly, methods such as electrocardiogram, echocardiogram, heart monitor, and heart magnetic resonance imaging are used. These methods require specialized medical device or heart monitoring devices, as well as the involvement of specialized personnel. Therefore, patients must visit a medical institution for testing, meaning the convenience of heart sound data detection is insufficient.

Secondly, in order to enhance convenience, smartwatches are used for data acquisition. However, the measurement results change depending on the position and tightness of the watch. Therefore, users cannot determine whether the measurement results output by the smartwatch are reliable.

Furthermore, the above method can only acquire one set of data at a time, and the data is easily affected by factors such as device placement and ambient noise. For example, if the electrodes of the device are not placed correctly or there are too many air bubbles during the pasting process, it may lead to poor electrode contact, incomplete signal acquisition, and data loss or incompleteness.

The present application provides a solution based on the acquisition function of two earplugs, which enables the heart sound data detecting device to obtain two sets of synchronous data. After analysis, if the analysis results of the two sets of data are consistent, it means that the two sets of data are valid and accurate. At this time, the corresponding analysis results can be output.

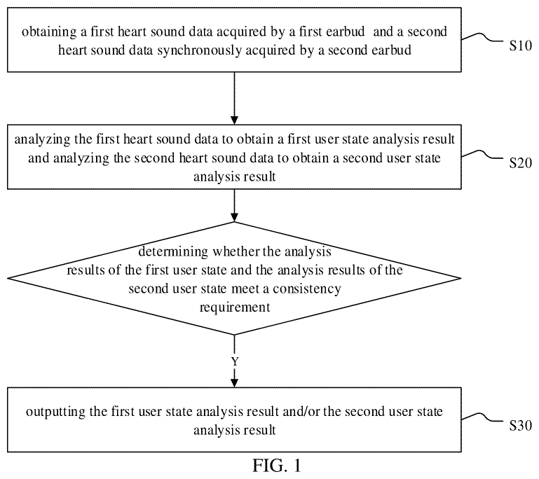

Based on this, an embodiment of the present application provides a method for controlling a heart sound data detecting device. Referring to FIG. 1, FIG. 1 is a flow chart a method for controlling a heart sound data detecting device according to an embodiment of the present application.

In this embodiment, the method for controlling the heart sound data detecting device includes steps S10 to S30:

Step S10, obtaining a first heart sound data acquired by a first earbud and a second heart sound data synchronously acquired by a second earbud.

It should be noted that the executing entity of this embodiment can be a computing service device with data processing, network communication and program running functions, or a heart sound data detecting device that can realize the above functions, such as a master control device, a cloud device, earplugs, etc., and the master control device includes a tablet computer, a personal computer, a mobile phone, etc.

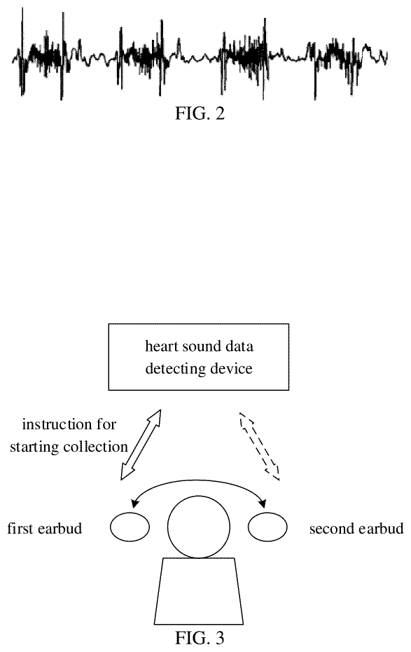

It should be noted that heart sound data refers to the signal data that reflects the sound produced by the mechanical movement of the heart, as shown in FIG. 2. Unlike traditional electrocardiogram devices, earbuds can be worn at all times and acquire heart sound data without complex operation, allowing users to monitor their heart health anytime, anywhere.

In order to ensure data accuracy and consistency, the first earbud and the second earbud need to work synchronously, starting and ending recording at the same time. This ensures that both devices capture heart sound data at the same time, facilitating subsequent data analysis and comparison.

In an embodiment, the heart sound data detecting device may actively send an instruction for starting acquisition to the first earplug and the second earplug to obtain the acquired heart sound data by the first earplug and the second earplug.

It should be noted that if the heart sound data detecting device only supports connecting to one earplug, then after the heart sound data is connected to the first earplug, it only needs to send an instruction for starting acquisition to the first earplug. After receiving the instruction for starting acquisition, the first earplug forwards the instruction for starting acquisition to the second earplug. Then the two earplugs will perform the acquisition action at the same time, and finally send the acquired first heart sound data and second heart sound data to the heart sound data detecting device in sequence. If the heart sound data detecting device supports connecting to two earplugs, a separate sending method can be used, that is, after the heart sound data detecting device establishes connections with the first earplug and the second earplug respectively, it sends an instruction for starting acquisition to the first earplug and the second earplug at the same time. Then the two earplugs will perform the acquisition action at the same time, and finally send the acquired first heart sound data and the second heart sound data to the heart sound data detecting device at the same time. This embodiment does not specifically limit the above two methods.

In addition, it should be noted that if the master control device can only communicate with one earbud at a time, and the default setting is that the master control device remains connected to the first earbud; in this scenario, the second earbud will first store the acquired second heart sound data in the local memory, and then send the second heart sound data to the first earbud after establishing a connection with the first earbud.

In an embodiment, the heart sound data detecting device runs an App control software, and functions of the App control software include: controlling the earbuds, viewing test results, viewing data calculated locally or on a cloud device, and conducting remote consultations with doctors. In daily use scenarios, the user inserts the first earbud into the left ear and the second earbud into the right ear. Then, the user opens the App control software on the heart sound data detecting device and clicks the “starting acquisition” icon, which triggers the instruction for starting acquisition and sends it to the first earbud, as shown in FIG. 3.

In an embodiment, a motion detecting device is mounted in a specific area of the heart sound data detecting device. When a specific action of the user is detected, such as double-clicking, long pressing, etc., the instruction for starting acquisition is generated and sent to the first earplug.

In an embodiment, the heart sound data detecting device has an automatic triggering function, and the automatic triggering function may be based on a built-in sensor, algorithm, or response to an external signal. When certain preset conditions are met, the heart sound data detecting device will automatically generate an instruction for starting acquisition and send it to the first earbud. In an embodiment, a specific time point or time period is set, and data acquisition is triggered when the time arrives, such as starting heart sound detecting at 6 pm every evening; using GPS or other positioning technology, data acquisition is triggered when the device or user enters or leaves a specific location, such as starting heart sound detecting once it is detected that the user has returned home. Data acquisition is triggered according to changes in the status of the user, such as when it is detected that the user's physiological parameters such as body temperature and respiratory rate exceed the normal range, heart sound detecting is immediately performed.

In another embodiment, the first earbud and the second earbud do not need to wait for instructions from the heart sound data detecting device. The first earbud and the second earbud will actively detect the heart sound data of the user, and once the detecting results are generated, they will actively send the acquired results to the heart sound data detecting device.

The above are merely two embodiments of the step S10 provided in this embodiment, and this embodiment does not specifically limit the specific embodiment of the step S10.

It can be understood that a single device or a single acquisition may have errors, such as equipment failure, environmental noise interference, etc. By acquiring two sets of data, the sample size is expanded, which can reduce errors caused by a single factor and improve data reliability.

Step S20, analyzing the first heart sound data to obtain a first user state analysis result and analyzing the second heart sound data to obtain a second user state analysis result.

Data analysis refers to further processing and interpretation of data to extract valuable analysis results, including but not limited to heart sound characteristics such as amplitude, frequency, and duration.

In an embodiment, the method includes inputting the first heart sound data and the second heart sound data into a data analysis model, analyzing the first heart sound data and the second heart sound data using machine learning algorithms, pattern recognition technology and other methods, and generating a corresponding first user status analysis result and a second user status analysis result.

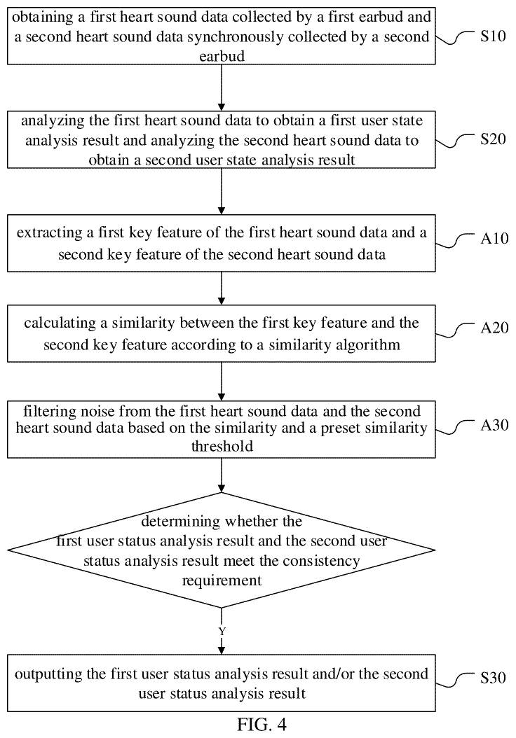

In addition, referring to FIG. 4, before the step S20, the method further includes:
Step A10, extracting a first key feature of the first heart sound data and a second key feature of the second heart sound data;
Step A20, calculating a similarity between the first key feature and the second key feature according to a similarity algorithm;
Step A30, filtering noise from the first heart sound data and the second heart sound data based on the similarity and a preset similarity threshold.

Data preprocessing refers to a series of processing steps performed on data before analysis, including but not limited to data conversion, data comparison, noise filtering, etc., so that subsequent analysis is more accurate and effective.

It is known that under normal circumstances, the heart sounds in the left ear and the right ear reflect the same heart activity, so the first heart sound data and the second heart sound data should have similar characteristics. If there is a significant difference, it means that there is noise caused by breathing, movement or other external noise.

In an embodiment, the heart sound data generally includes a plurality of heart cycles, and the heart sound data can be divided into several segments according to these cycles. The following explanation is given using the heart sound data of one cycle as an example.

Firstly, extracting a first key feature of the first heart sound data and a second key feature of the second heart sound data from at least one of the following dimensions.

Time domain feature extraction is as follows: extracting statistical features from time series data, such as mean, variance, peak, energy and other time domain features. Frequency domain feature extraction is as follows: converting the signal to the frequency domain through methods such as Fourier transform or wavelet transform, and extracting spectral features, such as frequency components, spectral morphology, etc. Filtering feature extraction is as follows: extracting the features of the filtered signal through the filter, such as the amplitude and phase after filtering. Transient feature extraction is as follows: identifying transient features in the signal, such as the heart sound activity cycle in heart sounds, etc.

Secondly, calculating the similarity between the first key feature and the second key feature using similarity algorithms such as Euclidean distance and cosine similarity.

Finally, if the similarity exceeds the threshold, they are considered similar. Otherwise, they are marked as having noise, and the noise is filtered out using filters, spectrum analysis, and other methods, or the heart sound data of this cycle is directly discarded, and retaining only valid data for subsequent data analysis.

Step S30, in response that the first user state analysis result and the second user state analysis result meet a consistency requirement, outputting the first user status analysis result and/or the second user status analysis result.

In an embodiment, plotting the dates of the first user status analysis result and the second user status analysis result into a histogram, and observing the shape, the center position, and the dispersion of the histogram to see whether the overall trends are consistent.

In another embodiment, calculating a correlation coefficient between the first user status analysis result and the second user status analysis result, such as a Pearson correlation coefficient, a Spearman correlation coefficient, etc., to evaluate the linear correlation between them.

It should be noted that the specific definition of consistency requirements may vary depending on the application scenario. In an embodiment, in some cases, if the difference between two results is less than a certain threshold, they are considered consistent; in other cases, the two results must match exactly to be considered consistent. This embodiment does not impose specific limitations.

If the first user state analysis result and the second user state analysis result meet the consistency requirement, it means that the two are mutually supportive and mutually verified, and both accurately reflect the same heart activity. Therefore, at least one analysis result can be output at this time.

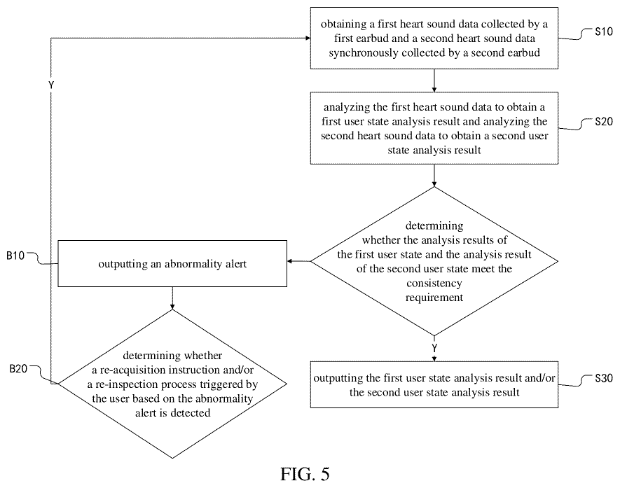

In addition, referring to FIG. 5, after the step S20, the method further includes:
Step B10, in response that the first user status analysis result and the second user status analysis result do not meet the consistency requirement, outputting an abnormality alert.
Step B20, in response detecting that a user triggers a re-acquisition instruction and/or a re-inspection process based on the abnormality alert, jumping to execute the obtaining the first heart sound data acquired by the first earbud and the second heart sound data synchronously acquired by the second earbud.

If the first user status analysis result and the second user status analysis result do not meet the consistency requirement, it means that there is an obvious difference or contradiction between the two, so it is necessary to output an abnormal reminder.

After seeing an abnormality alert, the user can choose to trigger a re-acquisition instruction. Accordingly, the heart sound data detecting device will re-acquire data after receiving the re-acquisition instruction of the user. Alternatively, if the consistency requirements are not met, the heart sound data detecting device will automatically trigger the re-check process and re-acquire data.

In this embodiment, the first earbud and the second earbud simultaneously acquire heart sound data, achieving dual verification of the same physiological signal. Only when the analysis results of the two are consistent will the data be considered valid. This mechanism ensures the reliability of the data. Furthermore, the user only needs to wear the earbuds throughout the entire testing process, eliminating the need to visit a medical facility. This ease of wear and operation greatly enhances the convenience of heart sound data testing.

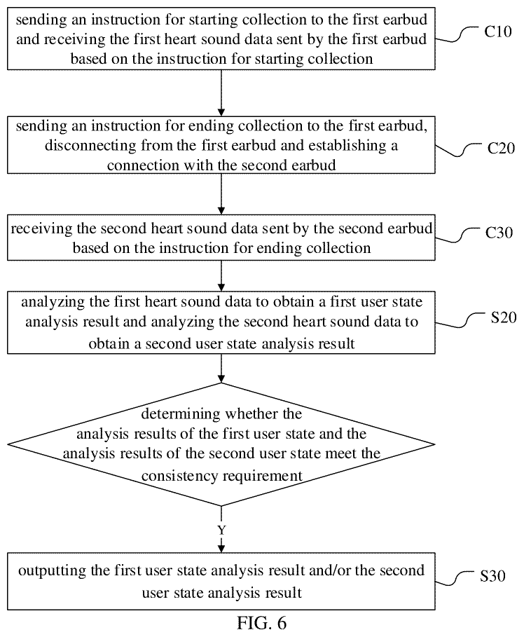

Based on the above embodiment of the present application, in another embodiment of the present application, the same or similar contents as those in the above embodiment can be referred to the above introduction and will not be repeated hereafter. On this basis, please refer to FIG. 6, the heart sound data detecting device is the master control device, and the step S10 includes steps C10 to C30:
Step C10, sending an instruction for starting acquisition to the first earbud and receiving the first heart sound data sent by the first earbud based on the instruction for starting acquisition.
Step C20, sending an instruction for ending acquisition to the first earbud, disconnecting from the first earbud and establishing a connection with the second earbud.
Step C30, receiving the second heart sound data sent by the second earbud based on the instruction for ending acquisition.

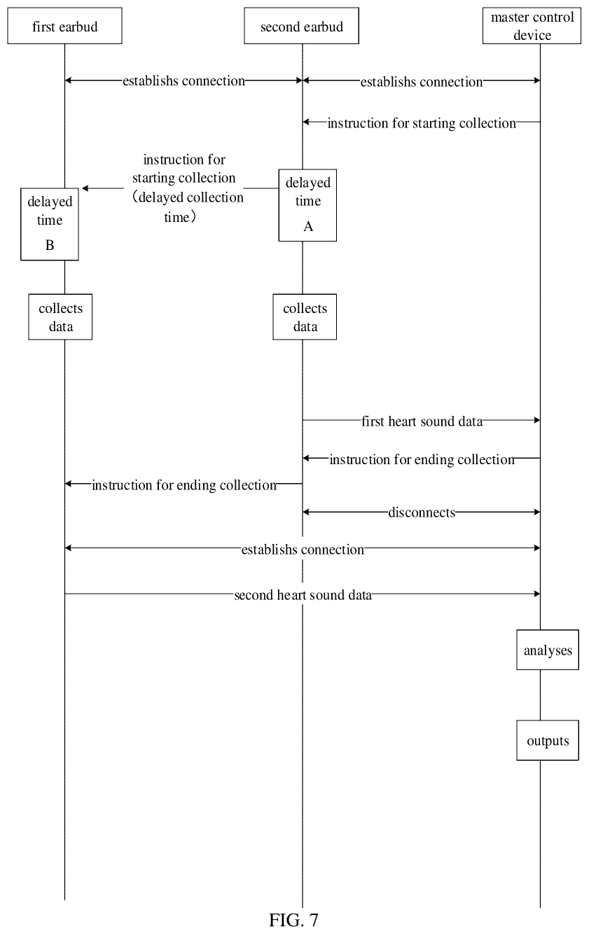

Refer to FIG. 7, FIG. 7 is a signaling flow chart of this embodiment.

It should be noted that the master control device includes, but is not limited to, a mobile phone, a tablet, or other master control terminal with wireless communication, display, and camera functions, and this embodiment does not impose any specific limitations.

In an embodiment, in order to ensure stable and reliable communication, the master control device is limited to communicating with only one earbud at a time, and by default, the master control device is connected to the first earbud. Since the first and second earbuds are typically used together, they are usually connected. This allows the first earbud to promptly forward data received from the master control device to the second earbud, achieving synchronized operation.

The master control device generates an instruction for starting acquisition based on the user operation and sends it to the first earplug, and then receives the first heart sound data returned by the first earplug. The specific principle is the same as that of the above embodiment and will not be repeated here.

After completing the above operations, the master control device also needs to obtain the second heart sound data for comparative analysis. Therefore, the master control device needs to generate an instruction for ending acquisition based on the user operation and send it to the first earbud, and then disconnect from the first earbud. This process mainly involves steps such as determining the communication protocol, sending a disconnection instruction, earbud response, disconnection and releasing resources.

At this point, the master control device can start the device search function to discover the surrounding second earbud and then establish a communication connection with the second earbud.

Because the first earbud forwards the instruction for starting acquisition to the second earbud, the second earbud can begin acquiring the second heart sound data of the user before establishing a connection with the master control device. This means that once the connection is established, the master control device can receive the second heart sound data acquired by the second earbud, ensuring the timeliness and accuracy of the data.

In this embodiment, when the heart sound data detecting device serves as the master control device, it sends an instruction for starting acquisition or an instruction for ending acquisition based on user input to control the progress of the entire detecting process. This allows users to precisely control the duration and frequency of data acquisition as needed. This precise control helps ensure that the acquired heart sound data is sufficiently accurate and representative, thereby improving the reliability of subsequent data analysis.

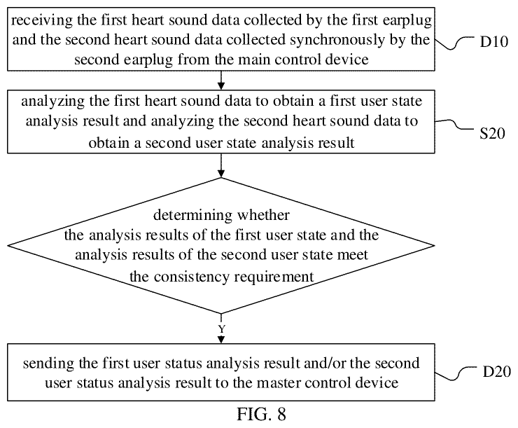

Based on the above embodiment of the present application, in another embodiment of the present application, the same or similar contents as those in the above embodiment can be referred to the above description and will not be repeated hereafter. On this basis, please refer to FIG. 8, the heart sound data detecting device is a cloud device, the step S10 includes step D10:

Step D10, receiving the first heart sound data acquired by the first earplug and the second heart sound data acquired synchronously by the second earplug from the master control device.

The step S30 includes step D20:

Step D20, in response that the first user status analysis result and the second user status analysis result meet the consistency requirement, sending the first user status analysis result and/or the second user status analysis result to the master control device.

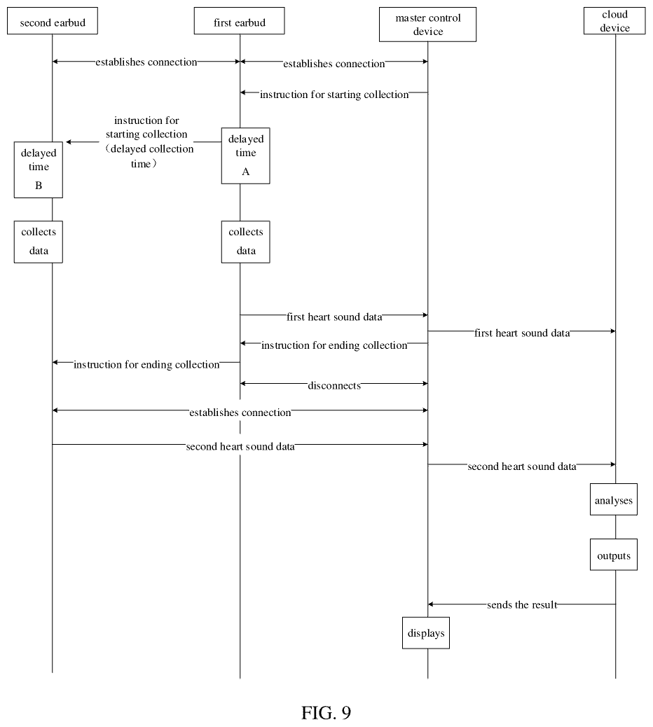

Refer to FIG. 9, FIG. 9 is a signaling flow chart of this embodiment.

It should be noted that a cloud device refers to a remote server or server cluster that utilizes cloud computing technology to receive, process, and store large amounts of data. The advantages of cloud devices lie in their powerful computing and data storage capabilities, as well as the flexibility and scalability enabled by cloud computing. Cloud devices can also facilitate remote consultations between users and doctors.

After the master control device receives the first heart sound data acquired by the first earbud and the second heart sound data acquired synchronously by the second earbud, the master control device will not process the data locally, but will forward the data to the cloud device. The processing principle is the same as the above embodiment and will not be repeated here.

Since the master control device is usually a device that users use daily, such as a smartphone, a tablet, or a computer, which has an intuitive user interface, the cloud device needs to send the analysis results to the master control device so that the user can view and understand the data and analysis results processed by the cloud device.

In this embodiment, data preprocessing and analysis are performed on cloud devices, fully leveraging their powerful computing resources and big data processing capabilities to improve data processing efficiency and accuracy. This also effectively reduces the burden on the master control device and improves the stability and reliability of the overall process.

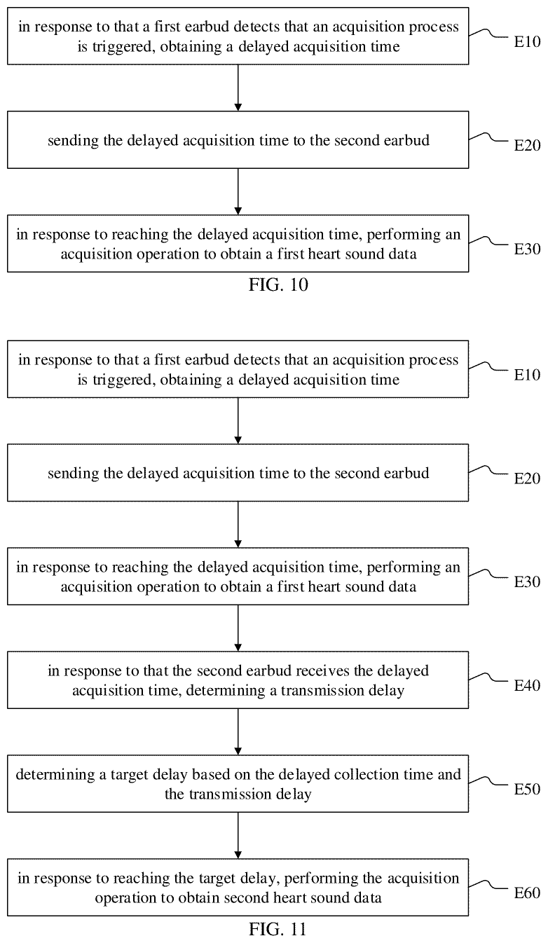

An embodiment of the present application provides a method for controlling a heart sound data detecting device. Referring to FIG. 10, FIG. 10 is a flow chart of the method for controlling the heart sound data detecting device according to another embodiment of the present application.

In this embodiment, the method for controlling the heart sound data detecting device includes steps E10 to E30:
Step E10, in response that a first earbud detects that an acquisition process is triggered, obtaining a delayed acquisition time.
Step E20, sending the delayed acquisition time to the second earbud.
Step E30, in response to reaching the delayed acquisition time, performing an acquisition operation to obtain a first heart sound data.

It should be noted that the earplug includes but is not limited to a microprocessor, a memory, a display module, a wireless communication module, a sound acquisition sensor module, etc.

The microprocessor can complete operations such as fetching instructions, executing instructions, and exchanging information with external memory and logic components, and it is the calculation and control part of the smart earplugs. The storage can store various applications and related data; the display module can display the display information of the system processing module, such as pictures, videos, UI, etc.; the wireless communication module can be but not limited to a Bluetooth module, a WiFi module, a 4G mobile communication module, etc., and the terminal device can be connected to an external device or network through this module; the sound acquisition sensor includes but is not limited to Microphone (MIC), acceleration sensor or Visual Processing Unit (VPU) and other sound data acquisition devices, which are placed in the ear canal of the user to acquire the heart sound signals of the user.

In an embodiment, when it is detected that the user clicks the “Start acquisition” icon on the APP control software, or the user makes a specific action in a specific area of the first earbud, or the time, position, status, etc. meet preset conditions, the first earbud will automatically trigger the acquisition process. The specific principle is the same as the above embodiment and will not be repeated here.

In an embodiment, once the data acquisition process is started, the first earbud will obtain the delayed data acquisition time A, the acquisition time A represents the delay from the current moment to the actual start of data acquisition, such as 100 ms.

On the one hand, referring to FIG. 9, the first earbud will send the delayed acquisition time A to the second earbud. In this way, after receiving the information from the first earbud, the second earbud can use this information to synchronize its own acquisition operation to ensure that both acquire heart sound data in the same time period.

On the other hand, once the delayed acquisition time A is set in the first earbud, a countdown function is activated. Once the countdown ends, that is, when the delayed acquisition time A is reached, the first earbud automatically begins acquiring data. After acquiring data for a certain period of time, or after detecting a command to end acquisition, data acquisition stops, and the first heart sound data is obtained.

In an embodiment, referring to FIG. 11, after the step E30, the method further includes:
Step E40, in response that the second earbud receives the delayed acquisition time, determining a transmission delay.
Step E50, determining a target delay based on the delayed acquisition time and the transmission delay.
Step E60, in response to reaching the target delay, performing the acquisition operation to obtain second heart sound data.

It can be understood that there is a certain time interval between sending and receiving data. Therefore, when the second earbud receives the delayed acquisition time sent by the first earbud, it is necessary to determine the transmission delay consumed therein.

In an embodiment, a first timestamp of the delayed acquisition time sent by the first earbud and a second timestamp of the delayed acquisition time received by the second earbud may be obtained by referring to a local real-time clock module or a network time. Then calculating according to the following formula: transmission delay=second timestamp−first timestamp.

In another embodiment, detecting the transmission distance and the transmission speed between the first earbud and the second earbud, and then calculating according to the following formula: transmission delay=transmission distance/transmission speed.

In an embodiment, obtaining the target delay B according to the following formula: target delay B=delayed acquisition time A−transmission delay.

At this point, the second earbud can set a countdown based on the target delay B. Once the countdown ends, that is, the target delay B is reached, automatic acquisition begins. After a certain period of acquisition, or after detecting the end of acquisition instruction, data acquisition stops and the second heart sound data is obtained.

It can be understood that if instruction forwarding delay is disregarded and two devices are allowed to collect data within the same countdown, the data from the two devices may not be fully synchronized or aligned due to network latency or other factors. By using different delay times, it is possible to ensure that the first earbud and the second earbud start data acquisition at the same time, thus avoiding data overlap or duplication, and thus reducing the resulting errors.

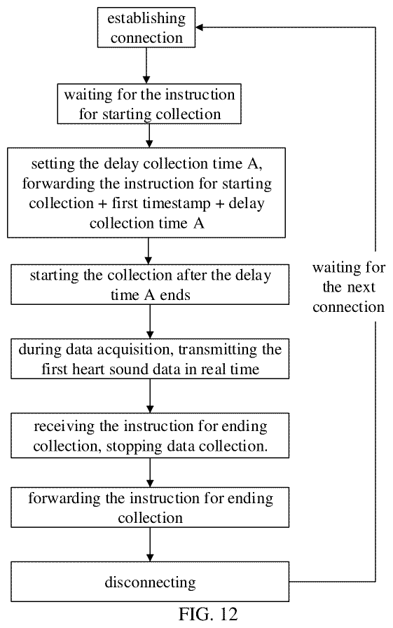

In an embodiment, in order to help understand the implementation process of the method for controlling the heart sound data detecting device obtained by combining this embodiment with the above embodiment, please refer to FIG. 12, FIG. 12 provides a schematic flow chart of the method for controlling the heart sound data detecting device. Specifically.
1. The user inserts the first earbud into the left ear and the second earbud into the right ear respectively, and opens the APP control software.
2. The user controls the software through the app and issues an instruction for starting acquisition. Upon receiving the instruction for starting acquisition, the first earbud obtains the first timestamp and the delayed acquisition time A, and forwards the instruction for starting acquisition, the first timestamp, and the delayed acquisition time A to the second earbud, synchronizing its clock and status with the second earbud.
3. After receiving the instruction for starting acquisition+the first timestamp+the delayed acquisition time A forwarded by the first earbud, the second earbud obtains its own second timestamp and calculates the target delay B.
4. After the countdown ends, the first earbud and second earbud simultaneously begin acquiring heart sound data from the left ear and the right ear. The first heart sound data is uploaded to the master control device in real time, and the second heart sound data is saved to the storage apparatus. Upon receiving the instruction for ending acquisition of the user, the first earbud stops data acquisition, sends the instruction for ending acquisition to the second earbud, and disconnects from the master control device.
5. After receiving the instruction for ending acquisition sent by the first earbud, the second earbud stops data acquisition, actively connects to the master control device, reads the saved heart sound data from the storage apparatus, and uploads it to the master control device.
6. After receiving the first and second heart sound data, the master control device will upload them to the cloud device for data analysis and processing.
7. After receiving the first heart sound data and the second heart sound data, the cloud device first compares and analyzes them, filters out noise data, and then performs data analysis on the first heart sound data and the second heart sound data separately to generate a first user status analysis result A and a second user status analysis result B. The cloud device compares the first user status analysis result A and the second user status analysis result B. If the results are consistent, the analysis result is sent to the master control device and displayed to the user. If the results are inconsistent, the data anomaly result is sent to the master control device and the user is requested to reacquire the data.

It should be noted that the above embodiments are only used to understand the present application and do not constitute a limitation on the method for controlling the heart sound data detecting device of the present application. More simple transformations based on this technical concept are all within the scope of protection of the present application.

The present application provides a heart sound data detecting device, and the heart sound data detecting device includes: at least one processor and a memory communicatively connected to the at least one processor. The memory stores instructions executable by the at least one processor, and the instructions are executed by the at least one processor so that the at least one processor can execute the method for controlling the heart sound data detecting device in the above embodiment.

The present application provides an earplug, and the earplug includes: at least one processor and a memory communicatively connected to the at least one processor. The memory stores instructions executable by the at least one processor, and the instructions are executed by the at least one processor so that the at least one processor can execute the method for controlling the heart sound data detecting device in the above embodiment.

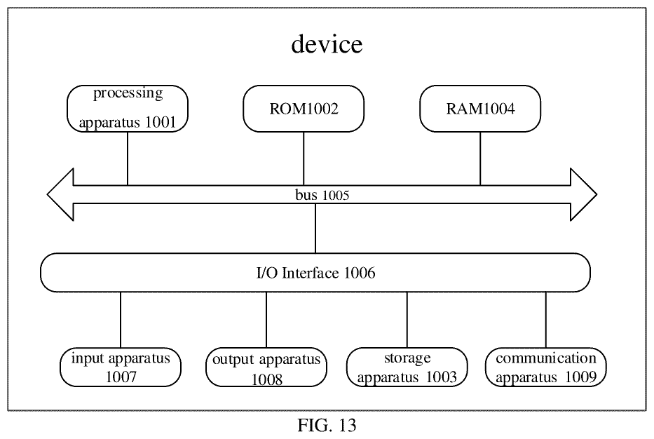

Referring to FIG. 13, FIG. 13 is a schematic structural diagram of a device suitable for implementing an embodiment of the present application. The devices in the embodiments of the present application may include, but are not limited to, mobile terminals such as a mobile phone, a laptop computer, a digital broadcast receiver, a Personal Digital Assistant (PDA), a Portable Application Description (PAD), a Portable Media Player (PMP), an in-vehicle terminal (e.g., in-vehicle navigation terminal), and fixed terminals such as a digital TV and a desktop computer. The device illustrated in FIG. 13 is merely an embodiment and should not limit the functionality or scope of use of the embodiments of the present application.

As shown in FIG. 13, the device may include a processing apparatus 1001 (e.g., a central processing unit, graphics processing unit, etc.), the processing apparatus 1001 can perform various appropriate actions and processes based on programs stored in the read-only memory (ROM) 1002 or programs loaded from the storage apparatus 1003 into the random access memory (RAM) 1004. The RAM 1004 also stores various programs and data required for device operation. The processing apparatus 1001, the ROM 1002 and the RAM 1004 are interconnected via bus 1005. The input/output (I/O) interface 1006 is also connected to the bus. Typically, the following systems may be connected to the I/O interface 1006: an input apparatus 1007 including, such as a touchscreen, a touchpad, a keyboard, mouse, an image sensor, a microphone, an accelerometer, a gyroscope, etc.; an output apparatus 1008 including, such as a liquid crystal display (LCD), a speaker, a vibrator, etc.; a storage apparatus 1003 including, such as a magnetic tape or a hard disk; and a communication apparatus 1009. The communication apparatus 1009 can allow the device to communicate with other devices wirelessly or wired to exchange data. Although the drawing shows a device with various systems, it should be understood that it is not required to implement or have all the systems shown. More or fewer systems can be implemented or have instead.

In particular, according to the embodiments disclosed in the present application, the processes described above with reference to the flowcharts can be implemented as computer software programs. For example, the embodiments disclosed in the present application include a computer program product including a computer program carried on a computer-readable medium, the computer program including program code for executing the method shown in the flowchart. In such an embodiment, the computer program can be downloaded and installed from a network via a communication apparatus, or installed from the storage apparatus 1003, or installed from the ROM 1002. When the computer program is executed by the processing apparatus 1001, the above functions defined in the method of the embodiment disclosed in the present application are executed.

The device provided in the present application, utilizing the method for controlling the heart sound data detecting device in the above embodiment, can solve the technical problem of inconvenient heart sound data detecting. Compared to the related art, the device provided in the present application offers the same beneficial effects as the method for controlling the heart sound data detecting device in the above embodiment. Other technical features of this device are the same as those disclosed in the above embodiment and are not further elaborated here.

It should be understood that the various parts disclosed in the present application can be implemented using a hardware, a software, a firmware, or a combination thereof. In the description of the above embodiment, specific features, structures, materials, or characteristics can be combined in any one or more embodiments or examples in a suitable manner.

The above description is merely a specific embodiment of the present application, but the scope of protection of the present application is not limited thereto. Any changes or substitutions that can be easily conceived by those person skilled in the art within the technical scope disclosed in the present application should be included in the scope of protection of the present application. Therefore, the scope of protection of the present application should be based on the scope of protection of the claims.

The present application provides a computer-readable storage medium, the computer-readable storage medium having computer-readable program instructions (i.e., a computer program) stored thereon, and the computer-readable program instructions are configured to execute the method for controlling the heart sound data detecting device in the above embodiment.

The computer-readable storage medium provided in the present application may be, for example, a USB flash drive, but is not limited to electrical, magnetic, optical, electromagnetic, infrared, or semiconductor systems, systems, or devices, or any combination thereof. More specific examples of computer-readable storage media may include, but are not limited to, an electrical connection having one or more wires, a portable computer disk, a hard disk, random access memory (RAM), read-only memory (ROM), erasable programmable read-only memory (EPROM or flash memory), optical fiber, a portable compact disk read-only memory (CD-ROM), an optical storage apparatus, a magnetic storage apparatus, or any suitable combination thereof. In this embodiment, the computer-readable storage medium may be any tangible medium that contains or stores a program that can be used by or in conjunction with an instruction execution system, system, or device. The program code contained on the computer-readable storage medium may be transmitted using any suitable medium, including, but not limited to, a wire, an optical cable, Radio Frequency (RF), etc., or any suitable combination thereof.

The computer-readable storage medium may be included in the device, or may exist independently without being incorporated into the device.

The above computer-readable storage medium carries one or more programs. When the above one or more programs are executed by the heart sound data detecting device, the heart sound data detecting device is configured to: obtain a first heart sound data acquired by the first earplug and a second heart sound data acquired synchronously by the second earplug; analyze the first heart sound data to obtain a first user state analysis result and analyze the second heart sound data to obtain a second user state analysis result; and in response to that the first user state analysis result and the second user state analysis result meet the consistency requirement, output the first user state analysis result and/or the second user state analysis result.

The computer-readable storage medium carries one or more programs. When the one or more programs are executed by the first wearable device, the first earbud is configured to: in response to that the first earbud detects that the acquisition process is triggered, obtain the delayed acquisition time; send the delayed acquisition time to the second earbud; and in response to reaching the delayed acquisition time, perform the acquisition operation to obtain the first heart sound data.

Computer program code for performing the operations of the present application may be written in one or more programming languages, or a combination thereof, including object-oriented programming languages such as Java, Smalltalk, C++, and conventional procedural programming languages such as “C” or similar programming languages. The program code may be executed entirely on computer of the user, partially on the computer of the user, as a stand-alone software package, partially on the computer of the user and partially on a remote computer, or entirely on a remote computer or a server. In the case of a remote computer, the remote computer may be connected to the computer of the user via any type of network, including a local area network (LAN) or a wide area network (WAN), or may be connected to an external computer (e.g., via the Internet using an Internet service provider).

The flowcharts and block diagrams in the accompanying drawings illustrate the possible architecture, functions and operations of the systems, methods and computer program products according to various embodiments of the present application. In this regard, each box in the flowchart or block diagram can represent a module, program segment or a part of code, and the module, program segment or a part of code contains one or more executable instructions for realizing the specified logical function. It should also be noted that in some alternative implementations, the functions marked in the box can also occur in a different order than that marked in the accompanying drawings. For example, two boxes represented in succession can actually be executed substantially in parallel, and they can sometimes be executed in the opposite order, depending on the functions involved. It should also be noted that each box in the block diagram and/or flowchart, and the combination of the boxes in the block diagram and/or flowchart, can be implemented by a dedicated hardware-based system that performs the specified function or operation, or can be implemented by a combination of dedicated hardware and computer instructions.

The modules described in the embodiments of the present application may be implemented in software or hardware, and the name of a module does not necessarily limit the unit itself.

The computer-readable storage medium provided in present application stores a computer-readable program instruction (i.e., a computer program) for executing the method for controlling the above heart sound data detecting device. This computer-readable storage medium can solve the technical problem of inconvenient heart sound data detecting. Compared to the related art, the beneficial effects of the computer-readable storage medium provided in the present application are similar to those of the method for controlling the heart sound data detecting device provided in the above embodiment, and are not further elaborated here.

The above descriptions are only embodiments of the present application, and are not intended to limit the scope of the present application. Under the inventive concept of the present application, any equivalent structural transformations made by using the contents of the description and drawings of the present application, or direct/indirect applications in other related technical fields are included in the scope of the present application.

What is claimed is:

1. A method for controlling a heart sound data detecting device, comprising:
obtaining a first heart sound data acquired by a first earbud and a second heart sound data synchronously acquired by a second earbud;
analyzing the first heart sound data to obtain a first user state analysis result and analyzing the second heart sound data to obtain a second user state analysis result; and
in response to that the first user state analysis result and the second user state analysis result meet a consistency requirement, outputting the first user state analysis result and/or the second user state analysis result.

2. The method according to claim 1, wherein the heart sound data detecting device is a master control device, and the obtaining the first heart sound data acquired by the first earbud and the second heart sound data acquired synchronously by the second earbud comprises:
sending an instruction for starting acquisition to the first earbud and receiving the first heart sound data sent by the first earbud based on the instruction for starting acquisition;
sending an instruction for ending acquisition to the first earbud, disconnecting from the first earbud and establishing a connection with the second earbud; and
receiving the second heart sound data sent by the second earbud based on the instruction for ending acquisition.

3. The method according to claim 1, wherein the heart sound data detecting device is a cloud device, and the obtaining the first heart sound data acquired by the first earbud and the second heart sound data acquired synchronously by the second earbud comprises:
receiving the first heart sound data acquired by the first earbud and the second heart sound data acquired synchronously by the second earbud from a master control device; and
the in response to that the first user state analysis result and the second user state analysis result meet the consistency requirement, outputting the first user state analysis result and/or the second user state analysis result comprises:
in response to that the first user state analysis result and the second user state analysis result meet the consistency requirement, sending the first user state analysis result and/or the second user state analysis result to the master control device.

4. The method according to claim 1, wherein after the analyzing the first heart sound data to obtain the first user state analysis result and analyzing the second heart sound data to obtain the second user state analysis result, the method further comprises at least one of the following steps:
in response to that the first user status analysis result and the second user status analysis result do not meet the consistency requirement, outputting an abnormality alert; and
in response to detecting that a user triggers a re-acquisition instruction and/or a re-inspection process based on the abnormality alert, jumping to execute the obtaining the first heart sound data acquired by the first earbud and the second heart sound data synchronously acquired by the second earbud.

5. The method according to claim 1, wherein before the analyzing the first heart sound data to obtain the first user state analysis result and analyzing the second heart sound data to obtain the second user state analysis result, the method further comprises:
extracting a first key feature of the first heart sound data and a second key feature of the second heart sound data;
calculating a similarity between the first key feature and the second key feature according to a similarity algorithm; and
filtering noise from the first heart sound data and the second heart sound data according to the similarity and a preset similarity threshold.

6. A method for controlling a heart sound data detecting device, comprising:
in response to that a first earbud detects that an acquisition process is triggered, obtaining a delayed acquisition time;
sending the delayed acquisition time to a second earbud; and
in response to reaching the delayed acquisition time, performing an acquisition operation to obtain a first heart sound data.

7. The method according to claim 6, further comprising:
in response to that the second earbud receives the delayed acquisition time, determining a transmission delay;
determining a target delay based on the delayed acquisition time and the transmission delay; and
in response to reaching the target delay, performing the acquisition operation to obtain second heart sound data.

8. A heart sound data detecting device, comprising: a memory, a processor, and a computer program stored in the memory and executable on the processor, wherein the computer program is configured to implement the method for controlling the heart sound data detecting device according to claim 1.

9. An earplug, comprising: a memory, a processor, and a computer program stored in the memory and executable on the processor, wherein the computer program is configured to implement the method for controlling the heart sound data detecting device according to claim 6.

10. A non-transitory computer-readable storage medium, wherein a computer program is stored on the non-transitory computer-readable storage medium, and when the computer program is executed by a processor, the method for controlling the heart sound data detecting device according to claim 1 is implemented.

11. A non-transitory computer-readable storage medium, wherein a computer program is stored on the non-transitory computer-readable storage medium, and when the computer program is executed by a processor, the method for controlling the heart sound data detecting device according to claim 6 is implemented.
